SEQUENCE LISTING (8 sequences)

SEQ ID NO: 1 - protein, 195 aa, Homo sapiens
MAAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYSDEEEPKDESARKND

SEQ ID NO: 2 - DNA, 1890 bp, Homo sapiens
gacccacgcgtccggggaggagaaagtggcgagttccggatccctgcctagcgcggcccaacctttactccagagatcatggctgccgaggatgtggtggcgactggcgccgacccaagcgatctggagagcggcgggctgctgcatgagattttcacgtcgccgctcaacctgctgctgcttggcctctgcatcttcctgctctacaagatcgtgcgcggggaccagccggcggccagcggcgacagcgacgacgacgagccgccccctctgccccgcctcaagcggcgcgacttcacccccgccgagctgcggcgcttcgacggcgtccaggacccgcgcatactcatggccatcaacggcaaggtgttcgatgtgaccaaaggccgcaaattctacgggcccgaggggccgtatggggtctttgctggaagagatgcatccaggggccttgccacattttgcctggataaggaagcactgaaggatgagtacgatgacctttctgacctcactgctgcccagcaggagactctgagtgactgggagtctcagttcactttcaagtatcatcacgtgggcaaactgctgaaggagggggaggagcccactgtgtactcagatgaggaagaaccaaaagatgagagtgcccggaaaaatgattaaagcattcagtggaagtatatctatttttgtattttgcaaaatcatttgtaacagtccactctgtctttaaaacatagtgattacaatatttagaaagttttgagcacttgctataagttttttaattaacatcactagtgacactaataaaattaacttcttagaatgcatgatgtgtttgtgtgtcacaaatccagaaagtgaactgcagtgctgtaatacacatgttaatactgtttttcttctatctgtagttagtacaggatgaatttaaatgtgtttttcctgagagacaaggaagacttgggtatttcccaaaacaggtaaaaatcttaaatgtgcaccaagagcaaaggatcaacttttagtcatgatgttctgtaaagacaacaaatccctttttttttctcaattgacttaactgcatgatttctgttttatctacctctaaagcaaatctgcagtgttccaaagactttggtatggattaagcgctgtccagtaacaaaatgaaatctcaaaacagagctcagctgcaaaaaagcatattttctgtgtttctggactgcactgttgtccttgccctcacatagacactcagacaccctcacaaacacagtagtctatagttaggattaaaataggatctgaacattcaaaagaaagctttggaaaaaaagagctggctggcctaaaaacctaaatatatgatgaagattgtaggactgtcttcccaagccccatgttcatggtggggcaatggttatttggttattttactcaattggttactctcatttgaaatgagggagggacatacagaataggaacaggtgtttgctctcctaagagccttcatgcacacccctgaaccacgaggaaacagtacagtcgctagtcaagtggtttttaaagtaaagtatattcataaggtaacagttattctgttgttataaaactatacccactgcaaaagtagtagtcaagtgtctaggtctttgatattgctcttttggttaacactaagcttaagtagactatacagttgtatgaatttgtaaaagtatatgaacacctagtgagatttcaaacttgtaattgtggttaaatagtcattgtattttcttgtgaactgtgttttatgattttacctcaaatcagaaaacaaaatgatgtgctttggtcagttaataaaaatggttttacccactaaaaaaaaaaaaa

SEQ ID NO: 3 - protein, 195 aa, Rattus norvegicus
MAAEDVVATGADPSELEGGGLLQEIFTSPLNLLLLGLCIFLLYKIVRGDQPGASGDNDDDEPPPLPRLKPRDFTPAELRRYDGVQDPRILMAINGKVFDVTKGRKFYGPEGPYGVFAGRDASRGLATFCLDKEALKDEYDDLSDLTPAQQETLNDWDSQFTFKYHHVGKLLKEGEEPTVYSDDEEPKDEAARKSD

SEQ ID NO: 4 - DNA, 678 bp, Rattus norvegicus
ctgcgaattcggcacgacgaggccgactgttccggatctctgcatagcagggcccaacctttgctccagagatcatggctgccgaggatgtggtggcgactggcgccgaccccagcgagctggagggcggcgggctgcttcaagagattttcacgtcgcctctcaacctgctgctccttggcctctgcatcttcctgctctacaagatcgttcgcggggaccagcccggtgccagtggggacaacgacgacgacgagccgcccccgctgccccgcctcaagccgcgtgacttcacccctgccgaactaaggcgatacgatggagtccaggacccgcgcattcttatggccatcaacggcaaggtgttcgacgtgaccaaaggccgcaagttctatgggccggaggggccatacggggtctttgctggaagagatgcatccaggggccttgccacattttgcctggacaaagaagcactgaaggatgagtatgatgacctttctgacctcactcctgcccagcaggagaccctgaatgactgggactctcagttcaccttcaagtaccatcacgtgggaaaactgctgaaggaaggggaggagccgactgtgtactcggatgatgaagaaccaaaagatgaggctgctcggaagagtgactgaagagtagtggagatat

SEQ ID NO: 5 - DNA, 21 bp, Artificial Sequence
  Description of Artificial Sequence Primer
tttgctccagagatcatggct

SEQ ID NO: 6 - DNA, 24 bp, Artificial Sequence
  Description of Artificial Sequence Primer
actactcttcagtcactcttccga

SEQ ID NO: 7 - DNA, 21 bp, Artificial Sequence
  Description of Artificial Sequence Primer
atcatggctgccgaggatgtg

SEQ ID NO: 8 - DNA, 23 bp, Artificial Sequence
  Description of Artificial Sequence Primer
tcatttttccgggcactctcatc

(19) United States
(12) Patent Application Publication
(10) Pub. No.: US 2004/0241840 A1
(43) Pub. Date: Dec. 2, 2004

(54) Compositions for therapy

(76) Inventor: Matthew Christopher Wright, Aberdeen (GB)

Correspondence Address: Bacon & Thomas, 4th Floor, 625 Slaters Lane, Alexandria, VA, 22314-1176, US

(21) Appl. No.: 10/490,101

(22) PCT Filed: Sep. 25, 2002
(86) PCT No.: PCT/GB02/04350

(30) Foreign Application Priority Data
Sep. 25, 2001 (GB) 0123063.0

(51) Int. Cl. C12N 5/08

(52) U.S. Cl. 435/370

(57) ABSTRACT

Provided are methods of inhibiting proliferation and/or trans-differentiation of hepatic stellate cells, which method comprises the step of contacting hepatic stellate cells with a ligand of the low affinity glucocorticoid binding site (LAGS), which may be the rat p28 receptor or the human hpr6.6 receptor. The invention also provides ligands for use in such methods (e.g. pregnenolone 16-alpha carbonitrile) and methods for screening for the same (e.g. based on competition or displacement assays using LAGS ligands such as dexamethasone). Such ligands may be used in the treatment of liver disorders.

### TECHNICAL FIELD

The present invention relates generally to methods and compositions for use in treatment of liver disease, particularly liver fibrosis.

### PRIOR ART

Liver fibrosis is characterised by an accumulation of extracellular matrix protein that with increasing severity, impairs normal function. It is a common response to hepatic damage mediated by a variety of mechanisms including xenobiotic damage (e.g. by drugs), viral infection (eg hepatitis B and C) and certain genetic diseases (eg hepatic hemochromatosis).

It is accepted that hepatic stellate cells (HSCs) play a central role in the development and resolution of liver fibrosis. HSCs are localised within the space of Disse and function to store retinoids in normal liver. In response to liver damage, HSCs “activate” to a myofibroblast-like (a-smooth muscle actin expressing) phenotype which is responsible for the majority of extracellular matrix protein deposition in liver fibrosis (for a review, see Friedman S L, J. Biol. Chem. 275, 2247-50 [2000]).

### DISCLOSURE OF THE INVENTION

The present inventor has now demonstrated that ligands of a specific saturable low affinity glucocorticoid binding site (LAGS) found in liver microsomes, which compete with dexamethasone at that site, surprisingly inhibit proliferation and trans-differentiation of hepatic stellate cells.

Thus ligands of LAGS may be used to modulate proliferation and differentiation of hepatic stellate cells and thus such ligands may find use in treatment of liver fibrosis.

In various aspects of the invention there are provided methods of inhibiting proliferation and/or trans-differentiation of hepatic stellate cells comprising the step of contacting hepatic stellate cells with a ligand of the low affinity glucocorticoid binding site (LAGS). By trans-differentiation, it is meant activation to an α-smooth muscle actin expressing cellular phenotype. The contacting step may be in vivo or in vitro, as described in more detail below.

Other aspects of the invention include methods of identifying novel inhibitors of differentiation of hepatic stellate cells, plus therapeutic compositions and uses thereof.

Some features and aspects of the invention will now be discussed in more detail.

LAGS Site

The LAGS site itself, and the biophysical characteristics which distinguish it from the glucocorticoid receptor, have previously been characterised. The following publications are concerned with the LAGS activity: Wright M. C., Paine A. J., Skett P. and Auld R. (1994). Induction of rat hepatic glucocorticoid-inducible cytochrome P450 3A by metyrapone. The Journal of Steroid Biochemistry and Molecular Biology 48, 271-276; Wright M. C. and Paine A. J. (1994). Induction of rat liver cytochrome P450 3A1 by metyrapone. In: Cytochrome P450, 8th International Conference (Ed. M. C. Lecher) pp 733-736. John Libbey Eurotext, Paris; Wright M. C. and Paine A. J. (1994). Induction of the cytochrome P450 3A subfamily correlates with the binding of inducers to a microsomal protein. Biochemical and Biophysical Research Communications 201, 273-279; Wright M. C. and Paine A. J. (1995). Characteristics of a membrane-associated steroid binding site in rat liver. Journal of Receptor Research 15, 543-556; Wright M. C., Maurel P. and Paine A. J. (1996). Metyrapone is a Cytochrome P450 3A inducer in Human Hepatocytes in vitro. Human and Experimental Toxicology 15, 203-204; Wright M. C., Wang X., Pimenta M., Ribeiro V., Paine A. J. and Lechner M. C. (1996). Glucocorticoid receptor-independent transcriptional induction of CYP3A1 by metyrapone and it's potentiation by glucocorticoid. Molecular Pharmacology 50, 856-863; Wright M. C., Allenby G. and Paine A. J. (1997). Effect of vitamin A deficiency on the expression of low affinity glucocorticoid binding site activity and glucocorticoid-dependent induction of CYP3A2 in rat liver. Biochemical and Biophysical Research Communications, 237, 211-216; Wright M. C., Edwards R., Pimenta M., Ribeiro V., Ratra G. S., Lechner M. C. and Paine A. J. (1997). Developmental changes in the constitutive and inducible expression of cytochrome P450 3A2. Biochemical Pharmacology 54, 841-846.

However its possible role in liver fibrosis had not previously been appreciated.

In separate work, a putative steroid (progesterone) binding site in rat and human liver has been cloned. It is termed ratp28 (rat) (Nolte et al, Biochim. Biophys. Acta, 1543 (2000) 123-150, Accession no AJ005837) and hpr6.6 (human) (Gerdes et al (1998) Biol. Chem. 379: 907-911, accession no Y12711) respectively. These sequences are set out in Annex I and II hereinafter. However, the function of such sites has yet to be elucidated.

As described in more detail below, the present inventors have shown that hepatic stellate cells express ratp28 and hpr6.6 in rats and humans respectively. Without being in any way limited to a particular theory or mechanism, it is believed that LAGS may correspond to ratp28 or human hpr6.6. Thus, in preferred embodiments of the invention, the low affinity glucocorticoid binding site is a receptor corresponding to the rat ratp28 receptor or the human hpr6.6 receptor and preferred ligands are ligands of the ratp28 receptor or the human hpr6.6 receptor.

The relevant contents of all these cited references, and accessions, inasmuch as they may be used by those skilled in the art in practising the present invention, are hereby specifically incorporated herein by reference.

Preferred Ligands

The ligand may be any ligand which binds the low affinity glucocorticoid binding site (LAGS) and which inhibits binding of a steroid such as dexamethasone to LAGS (an “anti-fibrogenic LAGS ligand”). In the light of the disclosure herein such ligands may be identified using any convenient assay, for example as described in Examples 1 and 2 herein.

The ligand, which may or may not be physiological or steroidal, is preferably an competitor of dexamethasone binding to the LAGS such as estrogen (oestradiol), pregnenolone 16-alpha carbonitrile (PCN), metyrapone or clotrimazole.

Non-naturally occurring and\or non-steroidal ligands may be preferred. For example, ligands may be optimised for highly specific binding and therapeutic use, while maintaining the LAGS binding affinity. Ligands according to the present invention may be provided isolated and\or purified from their natural environment, in substantially pure or homogeneous form, or free or substantially free of other biological material from the species of origin. Where used herein, the term “isolated” encompasses all of these possibilities.

Further, pharmaceutically acceptable active derivatives of such ligands and their use are within the scope of the present invention. Examples of such derivatives include, but are not limited to, salts, solvates, amides, esters, ethers, N-oxides, chemically protected forms, and prodrugs thereof.

Identification of Novel Modulators by use of LAGS Ligands

It is well known that pharmaceutical research leading to the identification of a new drug may involve the screening of very large numbers of candidate substances, both before and even after a lead compound has been found. This is one factor which makes pharmaceutical research very expensive and time-consuming. Means for assisting in the screening process can have considerable commercial importance and utility.

Generally speaking, those skilled in the art are well able to construct vectors and design protocols for recombinant gene expression of the LAGS binding site in cells or cell lines to facilitate screening, and the use of such cells and cell lines in the various identification process embodiments forms one aspect of the present invention. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, 1992.

Thus the present invention provides, in a further aspect, a method of screening for further substances which inhibit proliferation and/or trans-differentiation of hepatic stellate cells i.e. which inhibit or modulate low affinity glucocorticoid binding site (LAGS) activity, the method generally comprising comparing under comparable reaction conditions binding of a labelled LAGS ligand in the presence and absence of the test compound. LAGS ligands can be used to screen for substances in either a competitive or a displacement format.

Thus, in a further aspect of the present invention, a LAGS ligand is used in a method of screening for substances which affect, inhibit, modulate or mimic its activity or function with respect to LAGS

In this and other aspects, the substances (putative LAGS modulators) may be provided e.g. as the product of a combinatorial library such as are now well known in the art (see e.g. Newton (1997) Expert Opinion Therapeutic Patents,-7(10):-1183-1194).

Thus, in a displacement format, the invention provides a method for detecting the presence or amount of a putative LAGS modulator in a sample, the method comprising the steps of: (a) exposing the sample to a complex comprising a labelled LAGS ligand immobilised to a LAGS binding partner (e.g. the LAGS ligand binding site thereof, or an antibody raised to the LAGS modulator), (b) detecting any displaced labelled LAGS ligand.

In competitive formats, all the components of the assay are brought together simultaneously and the reduction in binding of the labelled LAGS ligand in the presence of the putative LAGS modulator is determined. In one embodiment, a binding constant Kd of the LAGS ligand for LAGS (or a preparation thereof) is determined by a saturation binding method in which increasing quantities of radiolabelled ligand are added to the LAGS, and the amount of labelled material bound at each concentration is determined. The appropriate binding equation describing the concentration of bound ligand as a function of the total ligand in equilibrium is fitted to the data to calculate the Bmax (the concentration of binding sites), and the Kd (which is approximately the concentration of the ligand required for half saturation of binding sites).

Reversibility of binding is a characteristic of ligands which, under equilibrium conditions, freely associate with and dissociate from their respective binding sites. Reversibility of binding of a specific compound is demonstrated by the labelled compound's ability to be displaced by unlabelled compound, after equilibrium binding of the labelled compound has been achieved.

To determine the binding constant of a test compound for a LAGS ligand binding site, the test compound is added, at increasing concentrations, to the LAGS preparation in the presence of a standard concentration of a radiolabeled LAGS ligand which exhibits reversible binding. The preparation is then rapidly filtered, washed and assayed for bound radiolabel. The binding constant (Kᵢ) of the test compound is determined from computer-fit competitive binding curves.

Essentially, methods of the present invention may be employed analogously to high throughput screens such as those well known in the art, and are based on binding partners—see e.g. WO 200011216 (Bristol-Myers Squibb), which enables fast, throughput screens for evaluation of test compounds that may modulate molecular targets whose specific nucleic acid or amino acid sequences are available, WO 200016231 (Navicyte), which describes a method of screening compound libraries by one or more bioavailability properties such as absorption (such a screen may be used in addition to or as an alternative to a receptor binding based screen); U.S. Pat. No. 6,027,873 (Genencor Intl.), which discloses a method of holding samples for analysis and an apparatus thereof and U.S. Pat. No. 6,007,690 (Aclara Biosciences), which describes integrated microfluidic devices which may be used in high throughput screens and other applications. Other high throughput screens are described in, for example, DE 19835071 (Carl Zeiss; F Hoffman-La Roche), WO 200003805 (CombiChem) and WO 200002899. (Biocept).

Optimisation of LAGS Ligands

Thus a substance identified using binding studies described above may be physiological or non-physiological in origin. Preferred anti-fibrogenic LAGS ligand may then be modified to enhance their binding to the LAGS receptor. For example, LAGS ligands based on steroid structures may be modified according to Example 5 in order to optimise binding.

Thus the present invention provides an iterative method of providing (designing and\or producing) an optimised anti-fibrogenic LAGS ligand, which method comprises the steps of:

(i) providing a steroidal LAGS ligand using the binding assay described above,

(ii) modifying the structure of the LAGS ligand as described herein to optimise its binding,

(iii) assessing the binding of the LAGS ligand using the binding assay described above.

Steps (ii) and (iii) may be repeated until no further optimisation is achieved.

The compounds may also be used to design of mimetics. This might be desirable where the active compound is difficult or expensive to synthesise or where it is unsuitable for a particular method of administration. There are several steps commonly taken in the design of a mimetic from a compound having a given target property. Firstly, the particular parts of the compound that are critical and/or important in determining the target property are determined. These parts or residues constituting the active region of the compound are known as its “pharmacophore”. Once the pharmacophore has been found, its structure is modelled according to its physical properties, e.g. stereochemistry, bonding, size and/or charge, using data from a range of sources, e.g. spectroscopic techniques, X-ray diffraction data and NMR. The three dimensional structure may be determined. Computational analysis, similarity mapping (which models the charge and/or volume of a pharmacophore, rather than the bonding between atoms) and other techniques can be used in this modeling process. A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound.

Therapeutic Compositions and their Use

LAGS ligands, either as disclosed herein or identified using methods as disclosed herein may be formulated into compositions for pharmaceutical and other uses.

Accordingly, a further aspect of the present invention provides a composition comprising a ligand of the low affinity glucocorticoid binding site (LAGS) of hepatic stellate cells for treating a patient with a liver disorder.

Diagnosis of a liver disorder in an individual may be by any of the criteria in accordance with agreed standards. Chronic liver damage of any aetiology may give rise to liver fibrosis and accordingly LAGS ligands may have potential in the treatment or therapy of any chronic liver disorder. In one embodiment ligands may be used in respect of cirrhosis (i.e. irreversible fibrosis which results in liver failure).

The terms “treatment” or “therapy” where used herein refer to any administration of a LAGS ligand e.g. an inhibitor of dexamethasone binding, or a salt thereof, intended to alleviate the severity of a disorder of the liver in a subject, and includes treatment intended to cure the disease, provide relief from the symptoms of the disease and to prevent or arrest the development of the disease in an individual at risk from developing the disease or an individual having symptoms indicating the development of the disease in that individual. Inhibitors of dexamethasone binding may be administered alone or in combination with other treatments, either simultaneously or sequentially dependent upon the condition to be treated.

Pharmaceutical compositions according to the present invention may comprise, in addition to the active compound (i.e. the LAGS ligand), a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration.

Compositions may be formulated for any suitable route and means of administration. Pharmaceutically acceptable carriers or diluents include those used in formulations suitable for oral, rectal, nasal, topical (including buccal and sublingual) or parenteral (including subcutaneous, intramuscular, intravenous, intradermal) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. The active compound (the inhibitor of dexamethasone binding) may be formulated as suppositories using, for example, polyalkylene glycols, acetylated triglycerides and the like, as the carrier. Liquid pharmaceutically administrable compositions can, for example, be prepared by dissolving, dispersing, etc, an active compound as defined above and optional pharmaceutical adjuvants in a carrier, such as, for example, water, saline, aqueous dextrose, glycerol, ethanol, and the like, to thereby form a solution or suspension.

Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound(s) in an amount effective to alleviate the symptoms of the subject being treated.

Dosage forms or compositions containing active ingredient in the range of 0.25 to 95% with the balance made up from non-toxic carrier may be prepared.

For oral administration, a pharmaceutically acceptable non-toxic composition is formed by the incorporation of any of the normally employed excipients, such as, for example, pharmaceutical grades of mannitol, lactose, cellulose, cellulose derivatives, sodium crosscarmellose, starch, magnesium stearate, sodium saccharin, talcum, glucose, sucrose, magnesium carbonate, and the like. Such compositions take the form of solutions, suspensions, tablets, pills, capsules, powders, sustained release formulations and the like. Such compositions may contain 1%-95% active ingredient, more preferably 2-50%, most preferably 5-8%.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like.

A more recently devised approach for parenteral administration employs the implantation of a slow-release or sustained-release system, such that a constant level of dosage is maintained. See, e.g., U.S. Pat. No. 3,710,795.

The percentage of active compound contained in such parental compositions is highly dependent on the specific nature thereof, as well as the activity of the compound and the needs of the subject. However, percentages of active ingredient of 0.1% to 10% in solution are employable, and will be higher if the composition is a solid which will be subsequently diluted to the above percentages. Preferably, the composition will comprise 0.2-2% of the active agent in solution.

For intravenous, cutaneous or subcutaneous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc.

A further aspect of the present invention provides a method of making a medicament for treating a liver disorder, the method comprising use of a ligand of the low affinity glucocorticoid binding site (LAGS) of hepatic stellate cells, eg admixing such a ligand with pharmaceutically acceptable components, as discussed above, to produce a pharmaceutical composition suitable for administration.

The invention also provides the use of a ligand of the low affinity glucocorticoid binding site (LAGS) of hepatic stellate cells in the preparation of a medicament for the treatment of a liver disorder

Effective Amount

In accordance with relevant aspects of the present invention the ligand of the low affinity glucocorticoid binding site (LAGS) or compositions comprising such ligands are intended to be administered to individuals. Administration is preferably in a “therapeutically effective amount”, this being sufficient to show benefit to a patient.

Therefore in a further aspect of the present invention, there is provided a method of treatment of a liver disorder, wherein said method comprises administering to a subject in need of treatment an effective amount of a ligand of the low affinity glucocorticoid binding site (LAGS) of hepatic stellate cells.

The actual (effective) amount administered, and rate and time-course of administration, will ultimately be at the discretion of the physician, taking into account the severity of the disease in a particular subject (e.g. a human patient or animal model) and the overall condition of the subject. Suitable dose ranges will typically be in the range of from 0.01 to 20 mg/kg/day, preferably from 0.1 to 10 mg/kg/day w

Further aspects and embodiments of the present invention, and modifications to those disclosed herein, will be apparent to persons skilled in the art. Illustration of embodiments of the present invention, by way of example, follows with reference to the figures. All documents mentioned herein are incorporated by reference.

### ANNEXES AND FIGURES

Annex I and II shows the sequences of hpr6.6 and ratp28 respectively.

FIG. 1: shows specific binding of dexamethasone to rat liver microsomes. Inset is shown a Scatchard plot of the data.

FIG. 2 illustrates competition of steroids and xenobiotics with dexamethasone for binding to the microsomes. LRₑ, ligand-receptor concentration at equilibrium; Lₑ, free ligand at equilibrium.

FIG. 3 illustrates the effect of LAGS ligands on α-smooth muscle actin expression in cultured human hepatic stellate cells.

FIG. 4 illustrates the result of RT-PCR analysis for ratp28 in rat hepatocytes and T6 cells.

FIG. 5 illustrates the results of RT-PCR analysis for hpr6.6 in human hepatic stellate cells.

FIG. 6 shows expression of target in COS-7 cells and detection of steroid binding activity in cell extracts. The percentage of cells that stained positive for beta galactosidase activity (grey bars) was determined in situ in separate wells by examining at least 5 radomly selected low power fields. Data are the mean and standard deviation of at least three separate determinations from the same experiment, typical of 2 separate experiments.

FIG. 7 shows the levels of binding of radiolabelled steroid in COS-7 cells transfected with pcDNA3.1e/ratp28. This was determined in whole cell extracts (control) or in the presence of the indicated concentration of unlabelled potential competitor. Control is the mean and standard deviation specific activity of 3 determinations from the same experiment after subtraction of non-specific binding. The percent of binding in the presence of unlabelled competitors was determined after subtraction of non-specific binding. Data are typical of at least 2 separate experiments.

### EXAMPLES

### Example 1

### A Low Affinity Glucocorticoid Binding Site (LAGS) can be Demonstrated in Liver Microsomes

Preparation of Liver Microsomes and Receptor Ligand Binding Studies.

Washed liver microsomes (100,000 g pellet) were prepared after canulating the portal vein of a rat and perfusing blood from the liver using ice-cooled hanks buffer. The liver was homogenised using a Potter homogenisor (Fisher Scientific, Loughborough, UK) in ice-cooled 20 mM Tris buffer pH 7.4 containing 100 mM KCl, 1 nM MgCl₂ and 250 mM sucrose and microsomes were pelleted from 20,000 g supernatants by standard methods (see Wright & Paine (1994) Biochem Biophys Res Comm 201).

Receptor Binding Studies—Dexamethasone

Receptor-steroid ligand binding studies were performed with resuspended microsomal protein (1-3mg protein/ml) using radiolabelled dexamethasone. Briefly, microsomes (2.5 mg protein/ml) were incubated to equilibrium at 4° C. (8 hrs) with varying concentrations of [³H]dexamethasone with or without 200-fold molar excess cold dexamethasone to determine non-specific binding (<5% of total) after removal of free ligand using dextran-charcoal. The addition of dextran-charcoal removes only free ligand and not non-specific unsaturable binding of ligand. Therefore, the co-incubation of 200 fold molar excess of cold ligand is used to displace specific saturable binding of radiolabelled ligand. The subtraction of the radioactive counts from dextran-charcoal treated samples containing excess unlabelled ligand from radioactive counts from dextran-charcoal samples containing only radiolabelled ligand was used to determine the amount of ligand bound to specific (saturable) receptors. Data were analysed using standard methods. The data was transformed for a Scatchard plot and the affinity constant (K_{D}) and receptor concentration [LRₑ]^{max} were determined by linear regression (Y upon X) from the slope and intersection with [LR]ₑ axis respectively.

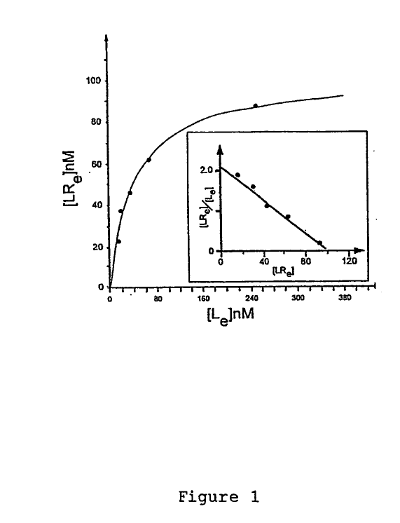

Receptor-steroid ligand binding studies identified a specific saturable receptor in the rat liver microsomes (FIG. 1). This “low affinity glucocorticoid binding site” (LAGS) was distinct from the glucocorticoid receptor on the basis of several biophysical characteristics that include significant differences in affinity for dexamethasone, lack of binding of glucocorticoid receptor antagonists (eg RU486) (Table 1) to LAGS and lack of sensitivity of steroid binding to the LAGS in the presence of the sodium arsenite (a sulfhydryl reagent) (see Wright & Paine (1995) J Recep & Signal Transduction Res 15: 543-556).

TABLE 1
effect of metyrapone and RU486 on the specific binding of
10 nM [³H]dexamethasone in rat liver cytosol (glucocorticoid
receptor) and microsomes (LAGS).

| | |
|---|---|
| | % specific binding compared |
| additions to | to dexamethasone alone |

| incubation | cytosol | microsomes |
|---|---|---|
| — | 100 +/− 7.7 | 100 +/− 10.5 |
| metyrapone | 105 +/− 4.0 | 57 +/− 13.9* |
| 10 μM RU486 | 0* | 123 +/− 9.2 |

Liver cell fractions were incubated with 10 nM [³H]dexamethasone with or without the indicated unlabelled compounds. Data are the mean and SD of 3 samples from the same experiment. Data are typical of 3 separate experiments. *Significantly different from control incubation P>5% using student's T test (2 tailed).

Receptor Binding Studies—Competition Analysis

The affinity of unlabelled ligands for the LAGS was assessed by competition analysis using a fixed radiolabelled concentration of 5×10⁻⁸M [³H] dexamethasone at 4° for 8 hours. Potential competitors were added from ethanol stocks such that the final ethanol concentration in the assay was less than 1% (v/v). Specific binding was determined using conventional methods as described above.

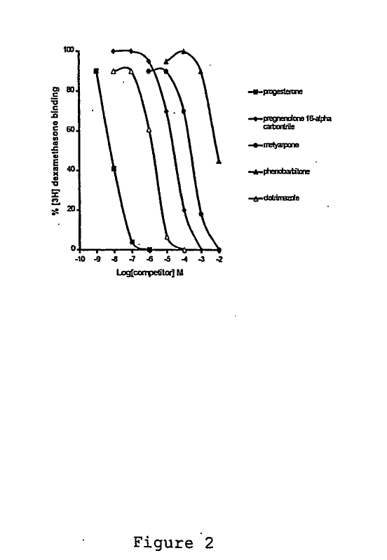

A number of structurally diverse compounds were found to compete with dexamethasone for binding to the LAGS (FIG. 2). These competitors included progesterone, pregnenolone 16-alpha carbonitrile (PCN)metyarpone, phenobarbitone and clotrimazole.

### Example 2

### LAGS Ligands Inhibit Hepatic Stellate Cell Growth and Differentiation

Rat hepatic stellate cells (HSCs) were isolated by pronase/collagenase perfusion and purified by isopycnic density centrifugation in Opti-prep™ (Nycomed) and elutriation (Wright et al (2001) Gastroenterology 121(3): 685-698). Human HSCs were isolated from discarded resected liver via a similar protocol. The use of human liver tissue for scientific investigation was approved by the UK South and West Local Research Ethics Commitee and was subject to patient consent.

HSCs were cultured in Dulbecco's modified Eagle Medium (DMEM) containing 4.5 g/l of glucose, supplemented with 16% (v/v) fetal calf serum, 80 u/ml penicillin, 80 μg/ml streptomycin and 32 μg/ml gentamycin. Cells were seeded onto plastic culture dishes and incubated at 37° C. in an humidified incubator gassed with 5% CO₂ in air. The culture medium was renewed after the first day and thereafter every 3-4 days. Rat and human HSCs were cultured for at least 14 days over which time they progressively increased the expression of a-smooth muscle actin expression from undetectable levels at isolation (data not shown).

Hepatic stellate cell isolation and culture under appropriate conditions results in a similar “activation” to the pro-fibrogenic phenotype in vivo (e.g. alpha smooth muscle and collagen I expression).

However, when rat and human HSCs were treated with ligands of LAGS, the proliferation and activation of the HSCs was inhibited. In addition, T6 cells (rat hepatic stellate cell line) were seeded at low density in 6 well plates in DMEM supplemented with 4% (v/v) FCS. After 1 hour, medium was changed and the cells were treated with drug as indicated in Table 1. The T6 cells were re-treated at 24 hours and counted at 48 hours. Human hepatic stellate cells were treated at days 2, 5, 7, 9 and 12 of culture and counted on day 15 of culture. Control cells were treated identically with 0.1% (v/v) ethanol used to prepare steroid stocks.

TABLE 2

% proliferation versus
control (100%)

| Treatment | T6 cells | human HSCs |
|---|---|---|
| 10 μM PCN | 55 +/− 17 | N/d |
| 5 μM clotrimazole | 18 +/− 4.7 | 25 +/− 13 |
| 500 μM metyrapone | 52 +/− 5.7 | 76 +/− 18 |
| 100 μM TAO | 106 +/− 2.8 | N/d |

PCN, pregneolone 16alpha carbonitrile, TAO, troleandomycin (this compound does not bind to the LAGS - see Wright & Paine (1994) Biochem Biophys Res Comm 201: 973-979), n/d, not determined.

As shown in Table 2, rat and human HSCs treated with ligands for the LAGS—such as clotrimazole, PCN and metyrapone—are inhibited in their ability to proliferate. Treatment with the high affinity physiological LAGS ligand progesterone or with dexamethasone had no effect on cell proliferation (results not shown).

The effect of LAGS ligands on activation to the pro-fibrogenic phenotype was investigated by measuring the effect of LAGS ligands on expression of α-smooth muscle actin in cultured human hepatic stellate cells. Briefly, Human HSCs (H10, 64 year old ♀) were cultured for 15 days and treated with the compounds: control, 0.1% v/v ethanol vehicle; oestradiol, 1 μM (also a ligand for the LAGS at this concentration—see Wright & Paine (1995) J Recep & Signal Transduction Res 15: 543-556); metyrapone, 200 μM or 1 μM clotrimazole. Whole cell extracts (10 μg protein/lane) were probed for alpha-smooth muscle actin and GFAP (glial fibrillary acidic protein—loading control) protein expression by Western blotting. Scanned blots were quantitated using Phoretix software (Nonlinear Dynamics, Newcastle, UK).

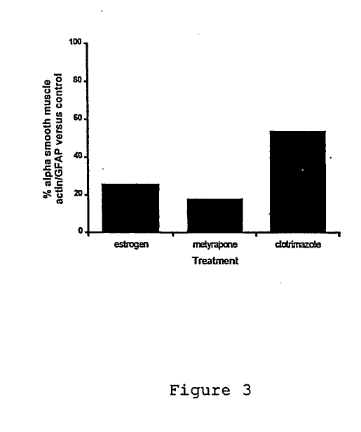

As shown in FIG. 3, oestradiol, metatyrapone and clotrimazole inhibited expression of α-smooth muscle actin by the hepatic stellate cells, thus demonstrating that these ligands inhibit trans-differentiation of these cells to the fibrogenic phenotype.

### Example 3

### Ratp28 and hpr6.6 are Expressed in Rat and Human Hepatic Stellate Cells Respectively

Total RNA was purified from HSCs using RNeasy kits (Qiagen, Southampton, UK) according to the manufacturer's instructions. For analysis of ratp28 mRNA expression, 4 μl of total RNA (approx 1 μg/μl) and 0.1 pmoles of the 3′ rLAGSDS PCR primer were heated together for 5 minutes at 90° C. and then placed on ice. First strand cDNA was then synthesized using MMLV reverse transciptase (Promega, Southampton, UK) and 1 mM dNTPs in total volume of 20 μl using supplier's protocol. DNA was then amplified using primers designed to hybridise the entire protein coding sequence (5′ rLAGSUS PCR primer sequence, 5′- TTTGCTCCAGAGATCATGGCT; 3′ rLAGSDS PCR primer sequence 5′- ACTACTCTTCAGTCACTCTTCCGA) through 35 cycles at 95° C. for 1 minute (denature), 52° C. for 1 minute (annealing) and 73° C. (elongation) for 2 minutes. Each 50 μl PCR reaction consisted of 5 μl of first-strand cDNA reaction, 1 mM dNTPs, 40 pmoles 5′ rLAGSUS PCR primer, 40 pmoles 3′ rLAGSDS PCR primer, 1×Pfu buffer (Promega, Southampton, UK) and lu Pfu polymerase (Promega, Southampton, UK). Hpr6.6 mRNA was examined in a similar fashion from human hepatic stellate cells using primers hLAGSUS PCR primer 5′ ATCATGGCTGCCGAGGATGTG and hLAGSDS PCR primer TCATTTTTCCGGGCACTCTCATC.

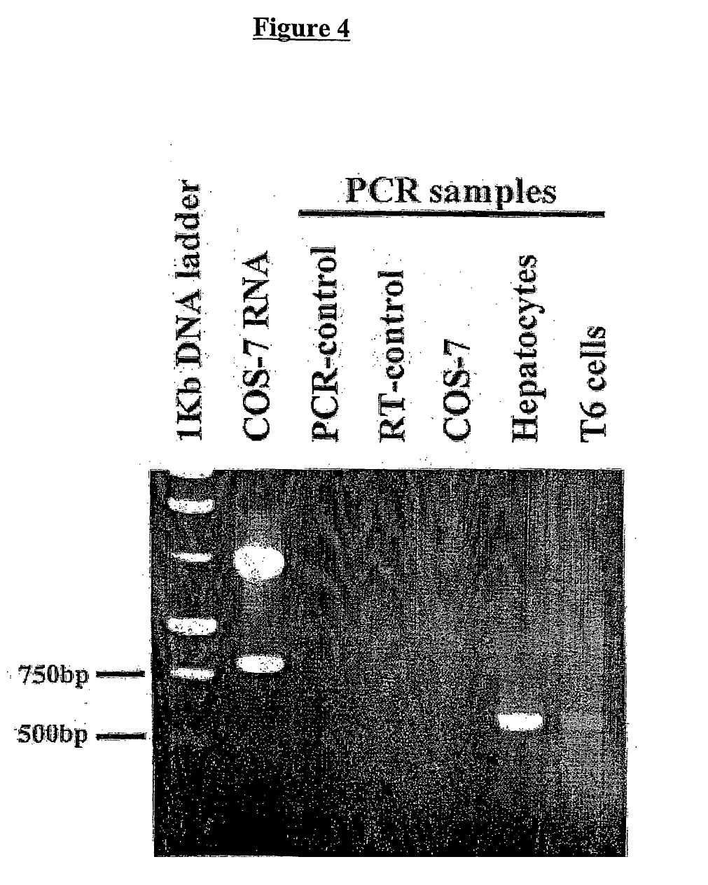

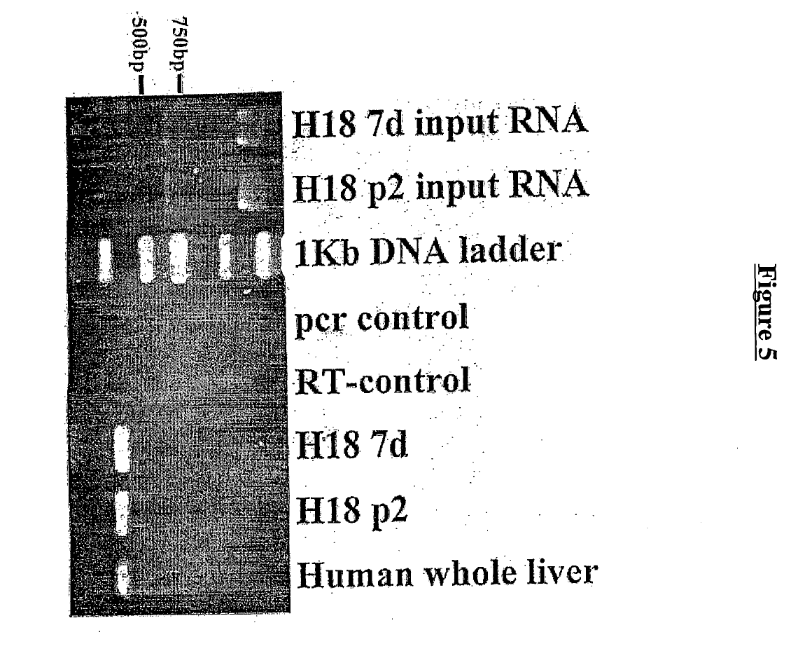

Fragments of the predicted size for ratp28 (FIG. 4) and hpr6.6 (FIG. 5) were readily amplified.

Fragments were analysed on 1% agarose gels containing ethidium bromide, excised and ligated into the pCR-BluntII-TOPO vector (Invitrogen) and transfected into One Shot (Invitrogen) cells using the manufacturer's instructions. Clones were isolated and plasmid DNA prepared and sequenced by standard methods.

The PCR fragments were found to be identical in sequence to the ratp28 (rats) or hpr6.6 (humans). HSCs therefore express a membrane associated steroid binding site. FIG. 4 demonstrates that the rat hepatic stellate cell line (T6 cells) express ratp28 with FIG. 5 demonstrating that cultured human HSCs express hpr6.6.

### Example 4

### Expression of ratp28 Results in a Binding Site with Binding Characteristics of LAGS

The ratp28 PCR product amplified as outlined above was inserted into pUniBlunt/V5-His TOPO vectors (Invitrogen, Paisley, Scotland) and transformed into PIR1 (Invitrogen, Paisley, Scotland) cells according to the manufacturer's instructions. Plasmids were restricted with XhoI/SacI to screen for inserts and selected plasmids sequenced. Correctly oriented pcr products cloned into pUniBlunt/V5-His-TOPO were sub-cloned into pcDNA3.1e using cre recombinase (Invitrogen, Paisley, Scotland) essentially according to manufacturer's instructions. Plasmids were transformed into TOP10 cells (Invitrogen, Paisley, Scotland) and clones initially screened for likely correct recombination by SacI restriction analysis. Selected clones were sequenced using the primer to ensure that the PCR insert was placed correctly downstream of the pcDNA3.1e promotor. COS7 cells were transfected at 30-50% confluency using Effectene transfection reagent (Qiagen, Southampton, UK) essentially according to the manufacturer's instructions with either pcDNA3.1e (control), pcDNA3.1e/lacZ or pcDNA3.1e/ratp28 (ratp28). Thirty hours after transfection, the medium was discarded and the cell washed twice with ice-cooled PBS. Cells were homogenised and specific binding of dexamethasone determined in extracts essentially as outlined for liver microsomes above.

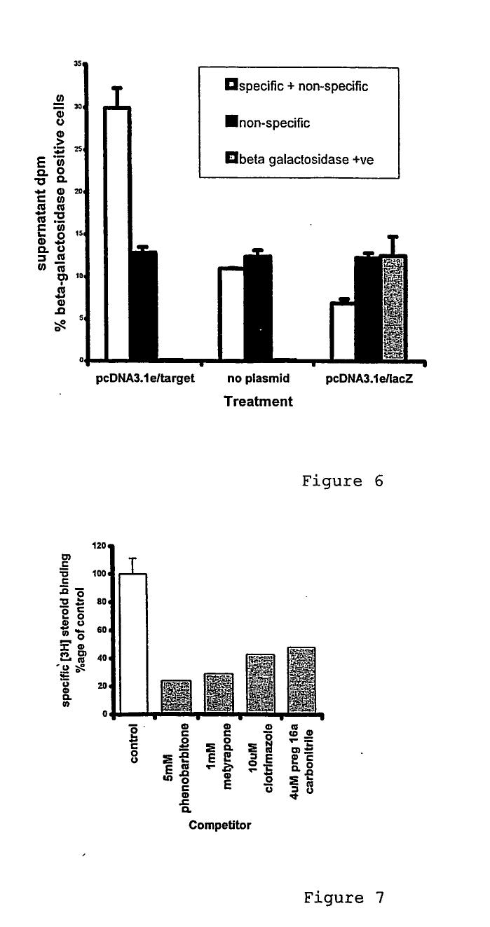

The results are shown in FIGS. 6 and 7, which are consistent with the competition studies carried out in Example 1 which used liver microsome LAGS.

### Example 5

### Ligand Structure-LAGS Binding Activity Data

In order to investigate optimisation and production of high affinity ligands for LAGS, a series of steroids containing the progesterone ring structure but with substitutions at various position were examined for their ability to compete with radiolabelled dexamethasone binding to the LAGS.

Binding studies were performed as outlined in Example 1. Non-specific binding of ligand was determined by co-incubating a paired sample with 1000-fold molar excess unlabelled dexamethasone. Non specific binding was typically less than 5% of specific +non-specific binding. Free ligand was removed using dextran-charcoal. The affinity of unlabelled ligands for the LAGS was assessed by competition analysis using a fixed radiolabelled concentration of 5×10⁻⁸M [³H] dexamethasone. Competitors were added from ethanol stocks such that final ethanol concentration in the assay was less than 1% (v/v).

The IC_{50%} concentration=the concentration of unlabelled competitor required to reduce the specific binding of radiolabelled dexamethasone to the LAGS to 50% of control.

Effect of substitution at position 3 of progesterone

| | Substitution | |
|---|---|---|
| Competitor | (R) | IC_{50%} |
| progesterone | O═ | 50 nM |
| 4 pregnene-3β-ol- | HO— | 500 nM |
| 20-one | | |
| 4 pregnene-3β-ol- | CH₃COO— | 500 nM |
| 20-one acetate | | |
| 4 pregnene-3β-ol- | HOOC—CH₂—O—N═ | >100 μM |
| 20-one | | |
| carboxymethyloxime | | |

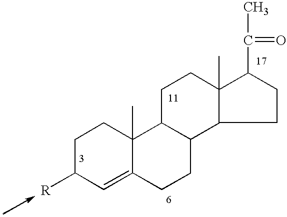

Effect of substitution at position 6 of progesterone

| | Substitution | |
|---|---|---|
| Competitor | (R) | IC_{50%} |
| progesterone | H— | 50 nM |
| 6α- | HO— | 5 μM |
| hydroxyprogesterone | | |
| 6β- | HO— | 10 μM |
| hydroxyprogesterone | | |
| 6β-progesterone | CH₃—COO— | 500 nM |
| acetate | | |

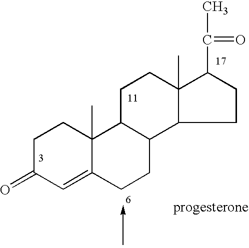

Effect of substitution at position 11 of progesterone

| | Substitution | |
|---|---|---|
| Competitor | (R) | IC_{50%} |
| progesterone | H— | 50 nM |
| 11α- | HO— | 100 nM |
| hydroxyprogesterone | | |
| 11β- | HO— | 50 nM |
| hydroxyprogesterone | | |
| 11α-progesterone | CH₃—COO— | 100 nM |
| acetate | | |
| 11β-progesterone | CH₃—Bz—SO₃— | 10 μM |
| tosylate | | |

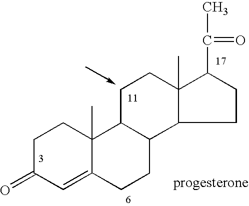

Effect of substitution at position 17 of progesterone

| | Substitution | |
|---|---|---|
| Competitor | (R) | IC_{50%} |
| progesterone | CH₃—CO— | 50 nM |
| androstenedione | O═ | 5 μM |
| 4 androstene-3- | CH₃OOC— | 100 nM |
| one 17β- | | |
| carboxylic acid | | |
| methyl ester | | |
| 4 androstene-3- | CH₃CH₂OOC— | 100 nM |
| one 17β- | | |
| carboxylic acid | | |
| ethyl ester | | |
| testosterone | CH₃CH₂COO— | 10 μM |
| proprionate | | |
| testosterone | HO— | 10 μM |
| 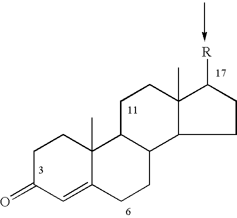 | | |

Effect of D ring substitution

| Competitor | Substitution (R) | IC_{50%} |
|---|---|---|
| dexamethasone | HO—CH₂—CO— | 50 nM |
| betamethasonenone | none - CH₃ at | 5 μM |
| | posn 16 is β | |
| | configuration | |
| 17β-carboxylic | HOOC— | 100 μM |
| acid | | |
| derivative* | | |
| biotinylated | biotin-CO— | >100 μM |
| dexamethasone | | |
| dexamethasone | CH₃—SO₂—O—CH₂—CO— | 10 μM |
| mesylate | | |

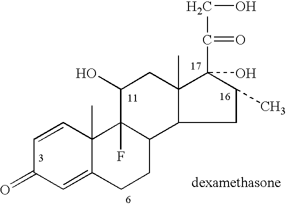

*9α-fluoro 16α-methyl-11β,17α-dihydroxy-1,4-pregn-3-one 17-carboxylic acid.

*9α-fluoro 16α-methyl-11β,17α-dihydroxy-1,4-pregn-3-one 17-carboxylic acid.

It can be seen that certain substitutions markedly affect the affinity of progesterone and dexamethasone for the LAGS. For example, the configuration of the methyl group at posn 16 appears to be important for dexamethasone since conversion from the alpha to beta configuration reduces the competition of the molecule by 100-fold.

Thus, where the LAGS ligand is steroidal, preferred compounds may be derivatives in which:

Position 3a and 3b selected from: each H—, 0═, CH₃COO— and H—

Position 6 selected from: H—, CH₃COO—,

Position 11 selected from: H—, HO—, CH₃COO—,

Position 17 selected from: CH₃CO—, CH₃OOC—, CH₃CH₂OOC—, HOCH₂CO—

More preferably:

Position 3: 0═

Position 6: H—

Position 11 selected from: H—, HO—,

Position 17 selected from: CH₃CO—, HOCH₂CO—

### Example 6

### Testing Pregnenolone 16alpha Carbontrile for Anti-Fibrotic Therapy in a Rat in vivo Model

Pregnenolone 16alpha carbonitrile (PCN) was chosen as a candidate drug to demonstrate the effect of LAGS ligands in inhibiting liver fibrosis in vivo.

Rats were administered CCl₄ twice weekly for 6 weeks to cause liver fibrosis and administered PCN once weekly during CCl₄ treatment.

Under the conditions of the test, there was no significant difference in liver serum (AST) enzyme levels or blinded histological scoring of haematoxylin and eosin (H&E) stained liver sections (both of which can be used to assess liver damage and necrosis), between CCl₄ and CCl₄/PCN treated rats (results not shown).

However PCN treatment significantly reduced the number of cells and the extent of α-smooth muscle actin staining in livers from rats treated with CCl₄ indicating a reduction in trans-differentiated pro-fibrogenic stellate cells. Sirius red staining (which detects collagens which constitute a significant proportion of fibrotic scarring) which was also shown to be significantly reduced in liver from rats treated with CCl₄ (results not shown).

Thus the compounds disclosed herein, and identifiable by the methods and screens disclosed herein, have particular utility in modulating (i.e. reducing) the fibrosis which results from liver damage e.g. resulting from disease or toxicity.

Annex I—sequence hpr6.6

Annex I-sequence hpr6.6

| | |
|---|---|
| LOCUS | NM_006667 1890 bp mRNA PRI 26-JUL-2001 |
| DEFINITION | Homo sapiens progesterone receptor membrane component 1 |
| | (PGRMC1), mRNA. |
| ACCESSION | NM_006667 |
| VERSION | NM_006667.2 GI:6857798 |
| KEYWORDS | . |
| SOURCE | human. |
| ORGANISM | Homo sapiens |
| | Eukaryota; Metazoa; Chordata; Craniata; Vertebrata; |
| | Euteleostomi; Mammalia; Eutheria; Primates; Catarrhini; |
| | Hominidae; Homo. |
| REFERENCE | 1 (bases 1 to 1690) |
| AUTHORS | Gerdes, D., Wehling, M., Leube, B. and Falkenstein, E. |
| TITLE | Cloning and tissue expression of two putative steroid membrane |
| | receptors |
| JOURNAL | Biol. Chem. 379 (7), 907-911 (1998) |
| MEDLINE | 98368853 |
| PUBMED | 9705155 |
| COMMENT | REVIEWED REFSEQ: This record has been curated by NCBI staff. |
| | The reference sequence was derived from Y12711.2. |
| | On Feb 3, 2000 this sequence version replaced gi:5729874. |
| | Summary: Progesterone binding protein is a putative steroid |
| | membrane receptor. The protein is expressed predominately in |
| | the liver and kidney. |
| | COMPLETENESS: complete on the 3′ end. |
| FEATURES | Location/Qualifiers |
| source | 1 . . . 1890 |
| | /organism = “Homo sapiens” |
| | /db_xref = “taxon:9606” |
| | /chromosome = “X” |
| | /map = “Xq22-q24” |
| gene | 1 . . . 1890 |
| | /gene = “PGRMC1” |
| | /note = “HPR6.6; MPR” |
| | /db_xref = “LocusID:10857” |
| CDS | 79 . . . 666 |
| | /gene = “PGRMC1” |
| | /codon_start = 1 |
| | /db_xref = “LocusID:10857” |
| | /product = “progesterone binding protein” |
| | /protein id = “NP_006658.1” |
| | /db_xref = “GI:5729875” |

/translation = “MAAEDVVATGADPSDLESGGLLHEIFTSPLNLLLLGLCIFLLYK
IVRGDQPAASGDSDDDEPPPLPRLKRRDFTPAELRRFDGVQDPRILMAINGKVFDVTK
GRKFYGPEGPYGVFAGRDASRGLATFCLDKEALKDEYDDLSDLTAAQQETLSDWESQF
TFKYHHVGKLLKEGEEPTVYSDEEEPKDESARKND”

| | |
|---|---|
| variation | 1633 |
| | /allele = “A” |
| | /allele = “C” |
| | /db_xref = “dbSNP:1051759” |
| variation | 1740 |
| | /allele = “A” |
| | /allele = “C” |
| | /db_xref = “dbSNP:1063649” |
| polyA_signal | 1856 . . . 1861 |
| polyA_site | 1877 |
| BASE COUNT | 546 a 397 c 427 g 520 t |
| ORIGIN | |

| | | | | | | |
|---|---|---|---|---|---|---|
| 1 | gacccacgcg | tccggggagg | agaaagtggc | gagttccgga | tccctgccta | gcgcggccca |
| 61 | acctttactc | cagagatcat | ggctgccgag | gatgtggtgg | cgactggcgc | cgacccaagc |
| 121 | gatctggaga | gcggcgggct | gctgcatgag | attttcacgt | cgccgctcaa | cctgctgctg |
| 181 | cttggcctct | gcatcttcct | gctctacaag | atcgtgcgcg | gggaccagcc | ggcggccagc |
| 241 | ggcgacagcg | acgacgacga | gccgccccct | ctgccccgcc | tcaagcggcg | cgacttcacc |
| 301 | cccgccgagc | tgcggcgctt | cgacggcgtc | caggacccgc | gcatactcat | ggccatcaac |
| 361 | ggcaaggtgt | tcgatgtgac | caaaggccgc | aaattctacg | ggcccgaggg | gccgtatggg |
| 421 | gtctttgctg | gaagagatgc | atccaggggc | cttgccacat | tttgcctgga | taaggaagca |
| 481 | ctgaaggatg | agtacgatga | cctttctgac | ctcactgctg | cccagcagga | gactctgagt |
| 541 | gactgggagt | ctcagttcac | tttcaagtat | catcacgtgg | gcaaactgct | gaaggagggg |
| 601 | gaggagccca | ctgtgtactc | agatgaggaa | gaaccaaaag | atgagagtgc | ccggaaaaat |
| 661 | gattaaagca | ttcagtggaa | gtatatctat | ttttgtattt | tgcaaaatca | tttgtaacag |
| 721 | tccactctgt | ctttaaaaca | tagtgattac | aatatttaga | aagttttgag | cacttgctat |
| 781 | aagtttttta | attaacatca | ctagtgacac | taataaaatt | aacttcttag | aatgcatgat |
| 841 | gtgtttgtgt | gtcacaaatc | cagaaagtga | actgcagtgc | tgtaatacac | atgttaatac |
| 901 | tgtttttctt | ctatctgtag | ttagtacagg | atgaatttaa | atgtgttttt | cctgagagac |
| 961 | aaggaagact | tgggtatttc | ccaaaacagg | taaaaatctt | aaatgtgcac | caagagcaaa |
| 1021 | ggatcaactt | ttagtcatga | tgttctgtaa | agacaacaaa | tccctttttt | tttctcaatt |
| 1081 | gacttaactg | catgatttct | gttttatcta | cctctaaagc | aaatctgcag | tgttccaaag |
| 1141 | actttggtat | ggattaagcg | ctgtccagta | acaaaatgaa | atctcaaaac | agagctcagc |
| 1201 | tgcaaaaaag | catattttct | gtgtttctgg | actgcactgt | tgtccttgcc | ctcacataga |
| 1261 | cactcagaca | ccctcacaaa | cacagtagtc | tatagttagg | attaaaatag | gatctgaaca |
| 1321 | ttcaaaagaa | agctttggaa | aaaaagagct | ggctggccta | aaaacctaaa | tatatgatga |
| 1381 | agattgtagg | actgtcttcc | caagccccat | gttcatggtg | gggcaatggt | tatttggtta |
| 1441 | ttttactcaa | ttggttactc | tcatttgaaa | tgagggaggg | acatacagaa | taggaacagg |
| 1501 | tgtttgctct | cctaagagcc | ttcatgcaca | cccctgaacc | acgaggaaac | agtacagtcg |
| 1561 | ctagtcaagt | ggtttttaaa | gtaaagtata | ttcataaggt | aacagttatt | ctgttgttat |
| 1621 | aaaactatac | ccactgcaaa | agtagtagtc | aagtgtctag | gtctttgata | ttgctctttt |
| 1681 | ggttaacact | aagcttaagt | agactataca | gttgtatgaa | tttgtaaaag | tatatgaaca |
| 1741 | cctagtgaga | tttcaaactt | gtaattgtgg | ttaaatagtc | attgtatttt | cttgtgaact |
| 1601 | gtgttttatg | attttacctc | aaatcagaaa | acaaaatgat | gtgctttggt | cagttaataa |
| 1861 | aaatggtttt | acccactaaa | aaaaaaaaaa | | | |

Annex II-sequence ratp28

| | |
|---|---|
| LOCUS | RNO5837 678 bp mRNA ROD 08-MAY-1998 |
| DEFINITION | Rattus norvegicus mRNA for putative progesterone binding |
| | protein. |
| ACCESSION | AJ005837 |
| VERSION | AJ005837.1 GI:3127856 |
| KEYWORDS | progesterone binding protein; putative. |
| SOURCE | Norway rat. |
| ORGANISM | Rattus norvegicus |
| | Eukaryota; Metazoa; Chordata; Craniata; Vertebrata; |
| | Euteleostomi; Mammalia; Eutheria; Rodentia; Sciurognathi; |
| | Muridae; Murinae; Rattus. |
| REFERENCE | 1 (bases 1 to 678) |
| AUTHORS | Noelte, I. |
| TITLE | Direct Submission |
| JOURNAL | Submitted (28-APR-1998) Noelte I., Biochemiezentrum |
| | Heidelberg, Inf |
| | 328, 69120 Heidelberg, GERMANY |
| REFERENCE | 2 (bases 1 to 678) |
| AUTHORS | Noelte, I., Sohn, K., Wegehingl, S. and Wieland, F. |
| TITLE | Rat homologue to a putative progesterone binding protein: |
| | molecular characterization and localization |
| JOURNAL | Unpublished |
| FEATURES | Location/Qualifiers |
| source | 1 . . . 678 |
| | /organism = “Rattus norvegicus” |
| | /strain = “fisher 344” |
| | /db_xref = “taxon:10116” |
| | /tissue_type = “liver” |
| gene | 75 . . . 662 |
| | /gene = “Lewi” |
| CDS | 75 . . . 662 |
| | /gene = “Lewi” |
| | /codon_start = 1 |
| | /product = “putative progesterone binding protein” |
| | /protein_id = “CAA06732.1” |
| | /db_xref = “GI:3127857” |
| | /db_xref = “SPTREMBL:O70606” |

/translation = “MAAEDVVATGADPSELEGGGLLQEIFTSPLNLLLLGLCIFLLYK
IVRGDQPGASGDNDDDEPPPLPRLKPRDFTPAELRRYDGVQDPRILMAINGKVFDVTK
GRKFYGPEGPYGVFAGRDASRGLATFCLDKEALKDEYDDLSDLTPAQQETLNDWDSQF
TFKYHHVGKLLKEGEEPTVYSDDEEPKDEAARKSD”

| | |
|---|---|
| misc_signal | 270 . . . 287 |
| | /gene = “Lewi” |
| | /function = “potential nuclear insertion signal” |
| misc_signal | 630 . . . 653 |
| | /gene = “Lewi” |
| | /function = “potential nuclear insertion signal” |
| BASE COUNT | 151 a 192 c 205 g 130 t |
| ORIGIN | |

| | | | | | | |
|---|---|---|---|---|---|---|
| 1 | ctgcgaattc | ggcacgacga | ggccgactgt | tccggatctc | tgcatagcag | ggcccaacct |
| 61 | ttgctccaga | gatcatggct | gccgaggatg | tggtggcgac | tggcgccgac | cccagcgagc |
| 121 | tggagggcgg | cgggctgctt | caagagattt | tcacgtcgcc | tctcaacctg | ctgctccttg |
| 181 | gcctctgcat | cttcctgctc | tacaagatcg | ttcgcgggga | ccagcccggt | gccagtgggg |
| 241 | acaacgacga | cgacgagccg | cccccgctgc | cccgcctcaa | gccgcgtgac | ttcacccctg |
| 301 | ccgaactaag | gcgatacgat | ggagtccagg | acccgcgcat | tcttatggcc | atcaacggca |
| 361 | aggtgttcga | cgtgaccaaa | ggccgcaagt | tctatgggcc | ggaggggcca | tacggggtct |
| 421 | ttgctggaag | agatgcatcc | aggggccttg | ccacattttg | cctggacaaa | gaagcactga |
| 481 | aggatgagta | tgatgacctt | tctgacctca | ctcctgccca | gcaggagacc | ctgaatgact |
| 541 | gggactctca | gttcaccttc | aagtaccatc | acgtgggaaa | actgctgaag | gaaggggagg |
| 601 | agccgactgt | gtactcggat | gatgaagaac | caaaagatga | ggctgctcgg | aagagtgact |
| 661 | gaagagtagt | ggagatat | | | | |

1. A method of inhibiting proliferation and/or trans-differentiation of hepatic stellate cells, which method comprises the step of contacting hepatic stellate cells with a ligand of the low affinity glucocorticoid binding site (LAGS).

2. A method as claimed in claim 1 wherein the LAGS is the rat ratp28 receptor or the human hpr6.6 receptor.

3. A method as claimed in claim 1 wherein the LAGS ligand is a LAGS antagonist.

4. A method as claimed in claim 1 wherein the LAGS ligand is non-naturally occurring.

5. A method as claimed in claim 1 wherein the LAGS ligand is non-steroidal.

6. A method as claimed in claim 1 wherein the LAGS ligand is pregnenolone 16-alpha carbonitrile or a pharmaceutically acceptable derivative of this.

7. A method as claimed in claim 1 which is carried out in vitro.

8. A method as claimed in claim 1 which is carried out in vivo.

9. A method as claimed in claim 8 for the treatment of a liver disorder, which method comprises administering to a subject in need of treatment an effective amount of a ligand of the low affinity glucocorticoid binding site (LAGS) of hepatic stellate cells.

10. A method as claimed in claim 9 wherein the liver disorder is cirrhosis.

11. A method of making a medicament for treating a liver disorder, the method comprising use of a ligand of the low affinity glucocorticoid binding site (LAGS) of hepatic stellate cells with a pharmaceutically acceptable excipient, carrier, buffer or stabiliser to produce a pharmaceutical composition suitable for use in a method as claimed in claim 8.

12. A pharmaceutical composition comprising a ligand of the low affinity glucocorticoid binding site (LAGS) of hepatic stellate cells for use in a method as claimed in claim 9.

13. (Cancelled)

14. A method of screening for a substance which inhibits proliferation and/or trans-differentiation of hepatic stellate cells, which method comprises assessing the binding of said substance to the low affinity glucocorticoid binding site (LAGS) of hepatic stellate cells.

15. A method as claimed in claim 14 which comprises comparing under comparable reaction conditions binding of ligand to the low affinity glucocorticold binding site (LAGS) of hepatic stellate cells in the presence and absence of the test substance.

16. A method as claimed in claim 14 which comprises the steps of:
(a) exposing a sample containing the putative inhibitor to a complex comprising a labelled LAGS ligand immobilised to a LAGS binding partner,
(b) detecting any displaced labelled LAGS ligand.

17. A method of producing a substance which inhibits proliferation and/or trans-differentiation of hepatic stellate cells, which method comprises:
(i) identifying the substance by use of the screening method of claim 14,
(ii) producing said substance.

18. A method of making a medicament for treating a liver disorder, the method comprising use of a ligand of the low affinity glucocorticoid binding site (LAGS) of hepatic stellate cells with a pharmaceutically acceptable excipient, carrier, buffer or stabiliser to produce a pharmaceutical composition suitable for use in a method as claimed in claim 9.
